(19) United States
(12) Patent Application Publication
(10) Pub. No.: US 2025/0099050 A1
(43) Pub. Date: Mar. 27, 2025

(54) COMPUTED TOMOGRAPHY SYSTEM, DETECTOR RING, AND METHOD FOR ADJUSTING AN APERTURE OPENING OF A COMPUTED TOMOGRAPHY SYSTEM

(71) Applicant: Siemens Healthineers AG, Forchheim (DE)

(72) Inventor: Hannes MOENIUS, Forchheim (DE)

(73) Assignee: Siemens Healthineers AG, Forchheim (DE)

(21) Appl. No.: 18/895,613

(22) Filed: Sep. 25, 2024

(30) Foreign Application Priority Data
Sep. 27, 2023 (EP) 23200069.5

(51) Int. Cl. A61B 6/40; A61B 6/00; A61B 6/03

(52) U.S. Cl. CPC A61B 6/4014; A61B 6/032; A61B 6/4435

(57) ABSTRACT

A computed tomography system comprises an examination region and a static gantry. The static gantry includes a detector ring and an X-ray source ring, which each encircle a substantially common axial direction. The X-ray source ring has a larger diameter than the detector ring. The detector ring is arranged offset in the axial direction with respect to the X-ray source ring such that a beam outlet opening of the X-ray source ring, when viewed in the radial direction with respect to the examination region, is, at least in part, not covered by the detector ring.

### CROSS-REFERENCE TO RELATED APPLICATION(S)

The present application claims priority under 35 U.S.C. § 119 to European Patent Application No. 23200069.5, filed Sep. 27, 2023, the entire contents of which is incorporated herein by reference.

### FIELD

One or more embodiments of the present invention relate to a computed tomography system, a detector ring for a computed tomography system and a method for adjusting an aperture opening of a computed tomography system.

### BACKGROUND

Independent of the grammatical term usage, individuals with male, female or other gender identities are included within the term.

Common computed tomography (CT) systems typically include an X-ray tube and a detector opposite the X-ray tube. In order to image a patient, the X-ray tube and detector rotate around the patient. A three-dimensional image of the patient can be captured due to the rotation. However, the rotation also has disadvantages. Due to the rotation, the requirements for the mechanics and data transmission are relatively high. These increased requirements lead to an increased effort in production and maintenance as well as generally to increased costs and can also impair the image quality. For example, there is the risk that vibrations arise due to the rotation, which can cause interfering frequencies in the measurement signals and thus artifacts in CT images. The rotation in general and any vibrations that are caused by the rotation in particular can lead to faster wear of the components. Furthermore, the rotations require that components have to be manufactured in a particularly solid manner, because a deformation of the components can have a major influence on the image quality. This ensures an increased space requirement and higher hardware costs. The data transmission, for example via sliding contacts, is also made more difficult by the rotation, as a result of which the capacity of the data transmission can also be limited.

### SUMMARY

It is an object of one or more embodiments of the present invention to provide a possibility with which the stated disadvantages can be addressed and at least partially improved.

At least this object is achieved by a computed tomography system, a detector ring and a method as claimed. Further features and advantages are evident in the dependent claims, the description and the attached figures.

In accordance with a first aspect of embodiments of the present invention, a computed tomography system is provided. The computed tomography system comprises an examination region and a static gantry. The static gantry comprises a detector ring and an X-ray source ring, which each enclose the examination region and essentially have a common axial direction. The X-ray source ring has a larger diameter than the detector ring, so that the X-ray source ring, when viewed in the radial direction, is arranged further out around the examination region than the detector ring. The detector ring is arranged offset in the axial direction with respect to the X-ray source ring in such a manner that a beam outlet opening of the X-ray source ring, when viewed in the radial direction with respect to the examination region, is at least in part not covered by the detector ring.

The examination region is in particular a region in which a subject or object can be examined via a computed tomography measurement that is performed by the computed tomography system, in that computed tomography (CT) images of the subject or object can be captured. The subject or object can be, for example, a human being or an animal or a body part, such as an organ or a body region, for example the chest region or head region, or another object (for example pieces of luggage or materials whose properties are to be examined).

The gantry, comprising the detector ring and the X-ray source ring, is arranged around the examination region. The gantry can in particular be a short annular tunnel. The axial direction of the annular tunnel can essentially correspond to the axial direction of the detector ring and the X-ray source ring. The axial direction can also be referred to as the z direction. In this context, the term “essentially” is to be understood in such a manner that certain deviations, for example, due to construction, can also be present. However, the axial directions of the components mentioned in each case, in particular the detector ring and the X-ray source ring, preferably do not deviate from one another by more than 10°. In this context, a radial direction is to be understood to be perpendicular to the axial direction and, in particular, to extend radially outward from a center point of the respective rings. Accordingly, the term radial direction is to be understood in such a manner that there are multiple directions that correspond to one radial direction, namely in accordance with the entirety of the possible radially extending directions. The radial direction can also be referred to as the x direction, y direction and/or x, y direction.

The gantry is static. This is to be understood in particular in such a manner that, in contrast to otherwise conventional gantries, no rotation of the gantry is provided during a CT measurement. This is rendered possible by the fact that a detector ring and an X-ray source ring are provided. In this manner, a complete image can be generated even without rotation of the detector and the X-ray source. In particular, the detector ring can be a 360° detector. In this sense, a 360° detector is a detector that can pick up signals that emanate from the examination region in all radial directions without having to rotate itself. In particular, the detector ring can be a static detector ring. In particular, the X-ray source ring can be a 360° X-ray emitter. In this sense, a 360° X-ray emitter is an X-ray emitter that can send X-rays from the entire 360° region around the examination region into the examination region without having to rotate itself. In particular, the X-ray source ring can be a static X-ray source ring. The detector ring can, for example, have multiple detector elements that are distributed around the circumference of the detector ring. The X-ray source ring can, for example, have multiple X-ray sources that are distributed around the circumference of the X-ray source ring. The X-ray sources can preferably be based on a nanotube field emission technique. X-ray sources that are based on field emission have the advantage over X-ray sources, which are usually used in CT systems of the prior art and which are based on heat-induced emission, in that a faster switchover can be rendered possible, since an afterglow effect can be avoided as far as possible. However, other X-ray sources can generally also be used. The X-ray source ring can be configured to successively activate individual or groups of a plurality of its X-ray sources during a CT scan of a subject or object in the examination region. As a result, a rotation or the advantage of a rotation can be quasi “simulated” in accordance with the CT systems that are customary in the prior art. Advantageously, an actual rotation of the gantry can thus be avoided, whereby the above-listed disadvantages of rotating parts can likewise be largely avoided. Advantageously, therefore, generally complex and expensive interfaces between parts that move relative to one another, such as sliding contacts, are not necessary. Rotational vibrations and resulting artifacts in the measurement data and wear can also be avoided. Furthermore, a more compact design is possible because less stable components are required. For example, module carriers and a detector mechanism of the detector or of the detector ring can be constructed in a significantly simpler, lighter and more cost-effective manner, since the detector does not have to endure any rotational forces. For example, it can be possible for a system in accordance with an embodiment of the present invention to also be configured for a mobile use. Furthermore, it can advantageously be possible to capture images more quickly and to be more flexible in the choice of successive capture angles, because it is not necessary to take into account inertia during mechanical rotation.

The X-ray source ring has a larger diameter than the detector ring. As a result, the X-ray source ring, when viewed in the radial direction, is arranged further out around the examination region than the detector ring. In other words, the X-ray source ring is arranged further out in the gantry than the detector ring. Advantageously, the X-ray source ring and the detector ring can thus be constructed in a particularly space-efficient manner. At the same time, the detector ring is arranged offset in the axial direction with respect to the X-ray source ring in such a manner that a beam outlet opening of the X-ray source ring, when viewed in the radial direction with respect to the examination region, is at least in part not covered, i.e. not entirely, by the detector ring. In particular, it is possible to provide that a center of the beam outlet opening is not covered by the detector ring. The beam outlet opening is to be understood in a broad sense. It generally refers to the location on the X-ray source ring at which X-rays are emitted from the X-ray sources to the outside. In some cases, for example for the purpose of collimation, in particular in accordance with some exemplary embodiments discussed herein, it can be advantageous to limit the beam exit in a targeted manner in part, in particular at the edge of the beam, via parts of the detector ring or parts on the detector ring. The detector ring can optionally serve as a collimator or part of a collimator for the beam outlet opening and thus cover a part of the beam outlet opening. However, it is provided that at least a part, preferably a center, of the beam outlet opening is generally not blocked.

In accordance with a preferred embodiment, the detector ring and the X-ray source ring partially overlap in the axial direction. This variant enables a particularly compact design of the gantry. Furthermore, the detector ring and the X-ray source ring can thus be assembled in a particularly tight manner in the axial direction, as a result of which an improved image quality can be achieved. In particular, a distance from a focus point of the X-ray sources of the X-ray source ring and the detector center can thus be reduced. As a result, for example, less pronounced image errors caused by an offset in the axial direction can occur, or image correction can be facilitated accordingly.

In accordance with one embodiment, the detector ring and the X-ray source ring are attached to the gantry from different sides, when viewed in the axial direction. For example, the detector ring can be mounted on the gantry in a positive axial direction (or in a +z direction), and the X-ray source ring can be mounted on the gantry in a negative axial direction (or in a −z direction). Due to the assembly from different sides, on the one hand, a particularly good spatial division can be possible, on the other hand, a particularly good accessibility of the two components, the X-ray source ring and the detector ring, in each case individually can be rendered possible. This can be advantageous, for example, for maintenance purposes. For example, the detector ring can be dismountable or partially dismountable without the X-ray source ring making dismounting significantly more difficult. For example, the detector ring can thus be directly accessible and removable, wherein for this purpose, for example, only one cover plate has to be removed.

In accordance with one embodiment, the gantry comprises an annular aperture having at least a first aperture part and a second aperture part, wherein the aperture is arranged in front of the beam outlet opening of the X-ray source ring, wherein the first aperture part is fastened to the detector ring or is an integral part of the detector ring. In particular, the aperture can be arranged in front of the beam outlet opening in such a manner that the aperture collimates the X-rays of the X-ray source ring. By fastening an aperture part to the detector ring, the detector ring can be arranged particularly close to the beam outlet opening, and an even more compact design can be achieved. The second aperture part can preferably be attached to the X-ray source ring or another part of the gantry or be an integral part thereof in each case. The aperture can be a ring that is completely closed in the circumferential direction of the X-ray source ring or the detector ring. The first aperture part and the second aperture part can together form a channel that limits the propagation of the X-ray radiation. The channel can in particular run in the radial direction. As seen in the circumferential direction, the channel can have an essentially constant cross-section and/or a constant channel opening with respect to the beam outlet opening.

In accordance with one embodiment, the detector ring is configured in such a manner that it can be displaced relative to the X-ray source ring in the axial direction together with the first aperture part, so that an aperture opening of the aperture can be adjusted by displacing the detector ring relative to the X-ray source ring in the axial direction. This embodiment can represent an efficient way to realize different aperture openings for the X-ray sources, in particular with regard to a distance along the axial direction between the first aperture part and the second aperture part. In particular, a particularly space-saving possibility of an adjustable aperture can thus be rendered possible. Advantageously, the position of the detector ring and the beam distribution can thus be adapted at the same time. In this manner, it can be possible to synchronize the propagation of the X-rays and the detector position in a simple and effective manner.

In accordance with one embodiment, the computed tomography system further comprises a patient couch, wherein the detector ring is attached to the gantry from the side of the patient couch in the axial direction. The patient couch can preferably be moved into and out of the gantry in the axial direction. A particularly simple accessibility to the detector ring, for example for maintenance purposes, can be rendered possible by attaching the detector ring in accordance with this embodiment. In particular, the detector ring or elements of the detector ring can be removed from the gantry in a particularly simple manner.

In accordance with one embodiment, the detector ring comprises functional units having detector elements, which can be removed individually from the gantry. Advantageously, in the case of individually removable functional units, for example, maintenance or replacement of individual detector elements can be possible without having to remove the entire detector ring. For example, it can thus be easier to maintain a general orientation and adjustment of the detector ring, so that only individual functional units need to be reinstalled, while the general orientation of the detector ring (and possibly of the aperture) can remain unchanged. This can simplify maintenance and make it more cost-effective. The detector ring can optionally comprise further functional units. Individual functional units can comprise or be modules, cables and/or DSF (data streaming front end). For example, a functional unit can be a module having a partially circular arrangement of detector elements.

In accordance with one embodiment, the detector ring comprises at least one housing section, wherein the housing section is configured in such a manner that at least one of the functional units can be inserted at least partially into the housing section. At least one functional unit can be inserted in the housing section. For example, it is possible to provide that the at least one functional unit is inserted into the housing section during operation of the detector ring. It can be provided that the functional unit is removed or can be removed from the housing for maintenance of the functional unit. The housing section can preferably be annular or partially annular. The housing section can in particular be configured in such a manner that the detector elements of at least one functional unit can be inserted into the housing section. Advantageously, the detector elements can thus have a position that is defined by the position of the housing section. In this manner, it can be possible to exchange detector elements without subsequently having to re-adjust their position, or it can be possible in a simplified manner to adjust the position of the detector elements. It can be provided that multiple functional units having detector elements can be inserted or are inserted in a housing section. For example, one housing section can be configured in such a manner that in each case two of the functional units can be inserted at least partially into the housing section. A usability of in each case two functional units can be particularly advantageous in order to enable both stable positioning and simple maintenance. The first aperture part as described herein can preferably be fastened to the housing section. It can thus preferably be prevented that a change in the position of the first aperture part takes place during the removal or replacement of the functional unit.

In accordance with one embodiment, the detector ring comprises multiple housing sections, which, when assembled, define the annular shape of the detector ring. In this sense, the housing sections can be segments, in particular circular segments, of the detector ring. The housing sections can be essentially equal-sized circular segments of the detector ring. The detector ring can preferably comprise three or more housing sections. Particularly preferably, the detector ring can comprise three housing sections. The three housing sections can be in particular essentially three equal-sized circular segments of the detector ring. For example, the housing sections can each be 120° segments. A division into three housing sections has proven to be particularly advantageous for maintenance purposes and a flexible adjustment of the orientation of the detector ring. The first aperture part as described herein can comprise a plurality of first aperture parts or can be assembled from a plurality of first aperture parts, which are each fastened to one of the housing sections.

In accordance with one embodiment, at least one of the functional units comprises a guide that is oriented so as to enable the functional unit to be inserted in the housing section in a precise manner in terms of positioning. Alternatively or additionally, the guide can be provided as part of the housing section.

The guide can be, for example, a guide rail or a guide rod. The housing section can, for example, comprise an inner edge or a guide tunnel that is configured so as to interact with the guide of the functional unit. The guide can be used to enable particularly precise positioning of the functional unit in the housing section. Furthermore, the guide can contribute to the fact that damage to the functional unit, in particular to the detector elements, for example due to impacts during insertion, can be prevented.

In accordance with one embodiment, the detector ring and/or the housing section comprises a scattered beam collimator, which is oriented to shield detector elements of the detector ring from scattered beams of the X-ray source ring, wherein the housing section is configured in such a manner that when the at least one functional unit is inserted into the housing section, the detector elements of the at least one functional unit are inserted in a precisely fitting manner with respect to the scattered beam collimator. Advantageously, a positioning of the scattered beam collimator can thus be maintained, while, for example, a functional unit is replaced.

In accordance with one embodiment, the X-ray source ring comprises multiple X-ray source segments. All advantages and features regarding the maintenance of the detector ring can be transferred analogously to the X-ray source ring. Thus, individual segments of the X-ray source ring can advantageously be removed. For example, corresponding functional units and/or housing sections of the X-ray source ring can also be provided.

A further aspect of embodiments of the present invention is a detector ring for a computed tomography system, wherein the detector ring comprises at least one annular or partially annular housing section and one or multiple functional units having detector elements, wherein the housing section is configured in such a manner that at least one of the functional units can be inserted at least partially into the housing section. The detector ring can in particular be a detector ring as described herein with regard to the computed tomography system. All advantages and features of the computed tomography system can be transferred analogously to the detector ring and vice versa. The detector ring can comprise multiple housing sections as described herein, which, when assembled, define the annular shape of the detector ring.

In accordance with one embodiment, at least one of the functional units comprises a guide that is oriented so as to enable the functional unit to be inserted in the housing section in a precise manner in terms of positioning. Alternatively or additionally, the guide can be provided as part of the housing section. The guide can be, for example, a guide rail or a guide rod. The housing section can, for example, comprise an inner edge or a guide tunnel that is configured so as to interact with the guide of the functional unit.

In accordance with one embodiment, the detector ring comprises a first aperture part, which is fastened to the detector ring or is an integral part of the detector ring, wherein the first aperture part is configured so as to produce, together with a second aperture part, an aperture for an X-ray emission ring, which has a larger diameter than the detector ring. In particular, the aperture can be provided for an X-ray ring as described herein. The first aperture part can be fastened for example to the housing section.

In accordance with one embodiment, the detector ring and/or the housing section comprises a scattered beam collimator, wherein the housing section is configured in such a manner that when the at least one functional unit is inserted into the housing section, the detector elements of the at least one functional unit are inserted in a precisely fitting manner with respect to the scattered beam collimator.

A further aspect of embodiments of the present invention is a method for adjusting an aperture opening of a computed tomography system having a static gantry, wherein the gantry comprises a detector ring and an X-ray source ring having a beam outlet opening and an annular aperture having at least a first aperture part and a second aperture part, wherein the aperture is arranged in front of the beam outlet opening of the X-ray source ring, wherein the first aperture part is fastened to the detector ring or is an integral part of the detector ring. The computed tomography system can in particular be a computed tomography system as described herein. The method comprises the following step:

displacing the detector ring and thus the first aperture part in the axial direction, so that a distance between the first aperture part and the second aperture part is changed and the aperture opening of the aperture is adjusted.

All advantages and features of the computed tomography system and the detector ring can be transferred analogously to the method and vice versa.

All embodiments described herein can be combined with one another, unless explicitly stated otherwise.

### BRIEF DESCRIPTION OF THE DRAWINGS

In the following, embodiments are described with reference to the attached figures.

FIG. 1 shows a schematic sectional view from the side of a computed tomography system in accordance with an embodiment of the present invention,

FIG. 2 shows a perspective view of a gantry of a computed tomography system in accordance with an embodiment of the present invention,

FIG. 3 shows a housing section and two functional units of a detector ring in accordance with an embodiment of the present invention and

FIG. 4 shows a schematic illustration of a method for adjusting an aperture opening of a computed tomography system having a static gantry in accordance with an embodiment of the present invention.

### DETAILED DESCRIPTION

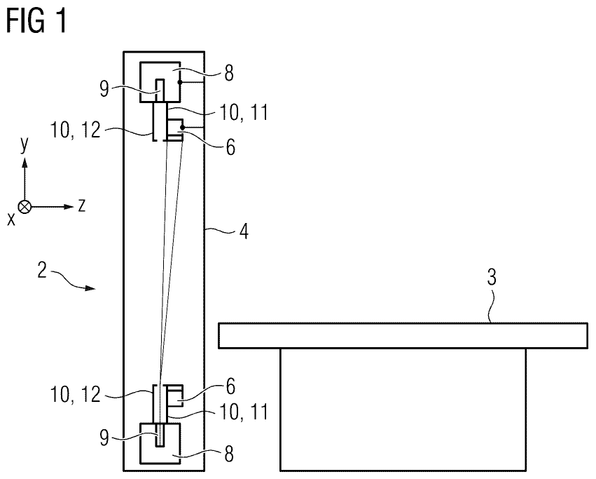

FIG. 1 illustrates a schematic sectional view from the side of a computed tomography system in accordance with an embodiment of the present invention. The computed tomography system comprises a static gantry 4, which is arranged as a short annular tunnel around an examination region 2. The gantry 4 comprises a detector ring 6 and an X-ray source ring 8, of which an upper part and a lower part are shown in each case and which each enclose the examination region 2. Detector ring 6 and X-ray source ring 8 have a common axial direction, which corresponds to the depicted z-direction. The detector ring 6 is attached to the gantry 4 from the right-hand side in relation to this illustration, and the X-ray source ring 8 is attached to the gantry 4 from the left-hand side. This means that the detector ring 6 and the X-ray source ring 8 are attached to the gantry 4 from different sides. In this case, the detector ring 6 is attached to the gantry 4 from the side on which a patient couch 3 is also provided. The detector ring 6 and the X-ray source ring 8 partially overlap in the axial direction, i.e. a part of the X-ray source ring 8 is in the same z-position as a part of the detector ring 6. The X-ray source ring 8 has a larger diameter than the detector ring 6, so that the X-ray source ring 8, when viewed in the radial direction in other words in the x, y-direction, is arranged further out around the examination region 2 than the detector ring 6. Moreover, the detector ring 6 is arranged offset in the axial direction with respect to the X-ray source ring 8 in such a manner that a beam outlet opening 9 of the X-ray source ring 8, when viewed in the radial direction with respect to the examination region 2, is not entirely covered by the detector ring 6. A first aperture part 11, which is part of an annular aperture 10 that is arranged in front of the beam outlet opening 9, is fastened to the detector ring 6. A second aperture part 12 of the aperture 10 is fastened to the X-ray source ring 8 itself. In this embodiment and preferably also in other embodiments, the aperture part 11 and the aperture part 12 are, in particular, independent assemblies, which, however, interact in terms of function. The detector ring 6 is configured in such a manner that it can be displaced in the axial direction (in other words here z-direction) together with the first aperture part 11, so that an aperture opening of the aperture 10 can be adjusted by displacing the detector ring 6 in the axial direction.

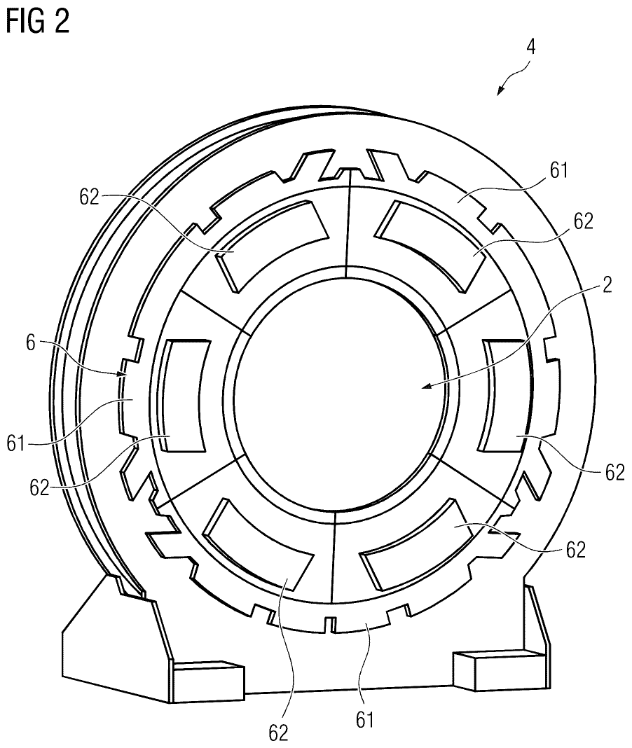

FIG. 2 illustrates a perspective view of a gantry 4 of a computed tomography system in accordance with an embodiment of the present invention. In this case, the gantry 4 is illustrated without an outer cladding. A detector ring 6 is attached to the gantry 4. An X-ray source ring 8 (not shown here) is provided on the opposite side of the gantry 4 (from this perspective, the rear side). The detector ring 6 comprises three partially annular housing sections 61 and, for each housing section 61, in each case two functional units 62 having detector elements 63, which are inserted in the housing section 61. The housing sections 61 together result in the annular shape of the detector ring 6. The functional units 62 can each be removed individually from the gantry 4. The X-ray source ring 8, which is not visible here, can optionally also comprise a plurality of X-ray source segments, analogous to the functional units 62 or housing sections 61 of the detector ring 6.

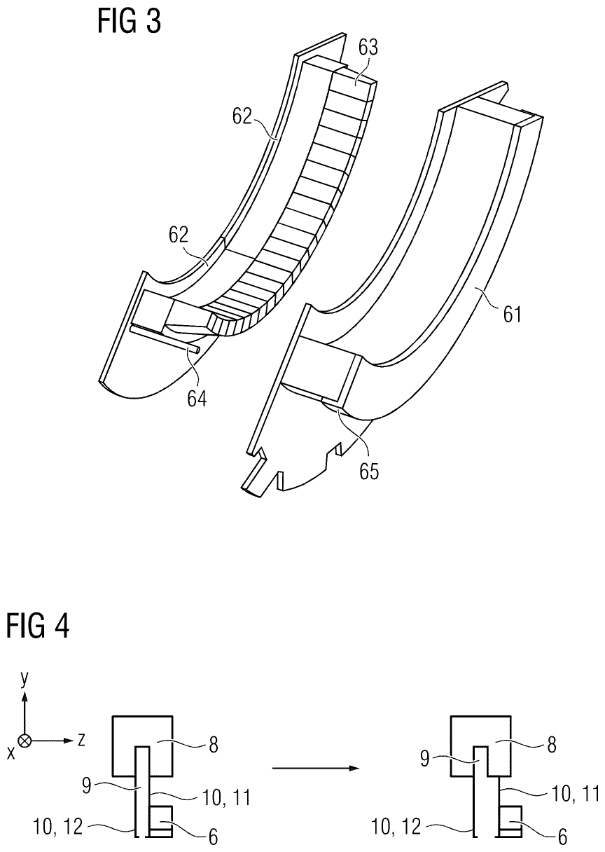

FIG. 3 illustrates a housing section 61 and two functional units 62 of a detector ring 6 in accordance with an embodiment of the present invention. The detector elements 63 of the two functional units 62 can be inserted into the housing section 61. The functional units 62 each comprise a guide 64 in the form of a guide rod, which is oriented so as to enable the functional units 62 to be inserted in the housing section 61 in a precise manner in terms of positioning, in that the guide 64 is guided along an inner edge of the housing section 61 and thus causes an orientation of the respective functional unit 62. The housing section 61 further comprises a scattered beam collimator 65, which is oriented to shield the detector elements 63 from scattered beams of the X-ray source ring 8. The guide 64 can also ensure that when the functional units 62 are inserted into the housing section 61, the detector elements 63 are inserted in a precisely fitting manner with respect to the scattered beam collimator 65.

FIG. 4 illustrates a schematic illustration of a method for adjusting an aperture opening of a computed tomography system having a static gantry 4 in accordance with an embodiment of the present invention. The gantry 4 comprises a detector ring 6 and an X-ray source ring 8 having a beam outlet opening 9 and an annular aperture 10 having a first aperture part 11 and a second aperture part 12, which is arranged in front of the beam outlet opening 9 of the X-ray source ring 8. The first aperture part 11 is fastened to the detector ring 6. As indicated, in the method the detector ring 6 and thus the first aperture part 11 is displaced in the axial direction (z-direction), so that a distance along the axial direction between the first aperture part 11 and the second aperture part 12 is changed (in this case enlarged) and the aperture opening of the aperture 10 is thereby adjusted.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

What is claimed is:

1. A computed tomography system comprising:
an examination region; and
a static gantry including a detector ring and an X-ray source ring,
wherein the detector ring and the X-ray source ring each enclose the examination region,
wherein the detector ring and the X-ray source ring each encircle a substantially common axial direction,
wherein the X-ray source ring has a larger diameter than the detector ring such that the X-ray source ring is arranged further out around the examination region than the detector ring when viewed in a radial direction, and
wherein the detector ring is arranged offset in the axial direction with respect to the X-ray source ring such that a beam outlet opening of the X-ray source ring is, at least in part, not covered by the detector ring when viewed in the radial direction with respect to the examination region.

2. The computed tomography system as claimed in claim 1,
wherein the detector ring and the X-ray source ring are attached to the static gantry at different sides when viewed in the axial direction.

3. The computed tomography system as claimed in claim 1, wherein
the static gantry includes an annular aperture having at least a first aperture part and a second aperture part,
the annular aperture is arranged in front of the beam outlet opening of the X-ray source ring, and
the first aperture part is fastened to the detector ring or is an integral part of the detector ring.

4. The computed tomography system as claimed in claim 3,
wherein the detector ring is configured to be displaced in the axial direction together with the first aperture part to adjust an aperture opening of the annular aperture.

5. The computed tomography system as claimed in claim 3, wherein
the second aperture part is attached to the X-ray source ring or to another part of the static gantry, or
the second aperture part is an integral part of the X-ray source ring or another part of the static gantry.

6. The computed tomography system as claimed in claim 1, wherein the detector ring and the X-ray source ring partially overlap in the axial direction.

7. The computed tomography system as claimed in claim 1, wherein
the computed tomography system further includes a patient couch configured to move into and out of the static gantry in the axial direction, and
the detector ring is attached to the static gantry at a side of the patient couch in the axial direction.

8. The computed tomography system as claimed in claim 1,
wherein the detector ring comprises functional units having detector elements, the detector elements being configured to be removed from the static gantry individually.

9. The computed tomography system as claimed in claim 8,
wherein
the detector ring includes at least one annular or partially annular housing section, and
the housing section is configured such that at least one of the functional units is insertable at least partially into the housing section.

10. The computed tomography system as claimed in claim 9,
wherein the at least one of the functional units includes a guide that is oriented to enable the at least one of the functional units to be inserted in the housing section in a precise manner in terms of positioning.

11. The computed tomography system as claimed in claim 9, wherein
the housing section includes a scattered beam collimator that is oriented to shield detector elements of the detector ring from scattered beams of the X-ray source ring, and
the housing section is configured such that when the at least one of the functional units is inserted into the housing section, the detector elements of the at least one of the functional units are inserted in a fitting manner with respect to the scattered beam collimator.

12. The computed tomography system as claimed in claim 9,
wherein
the detector ring includes multiple housing sections, and
the multiple housing sections, when assembled, define an annular shape of the detector ring.

13. A detector ring for the computed tomography system as claimed in claim 1, wherein the detector ring comprises:
at least one annular or partially annular housing section; and
one or more functional units including detector elements;
wherein the housing section is configured such that at least one of the one or more functional units is insertable at least partially into the housing section.

14. The detector ring as claimed in claim 13, wherein
the detector ring includes a first aperture part, the first aperture part being fastened to the detector ring or being an integral part of the detector ring, and
the first aperture part is configured to form, together with a second aperture part, an aperture for an X-ray emission ring, which has a larger diameter than the detector ring.

15. A method for adjusting an aperture opening of the computed tomography system as claimed in claim 1, wherein the X-ray source ring has the beam outlet opening and an annular aperture, wherein the annular aperture has at least a first aperture part and a second aperture part, wherein the annular aperture is arranged in front of the beam outlet opening, wherein the first aperture part is fastened to the detector ring or is an integral part of the detector ring, and wherein the method comprises:
displacing the detector ring and the first aperture part in the axial direction to change a distance between the first aperture part and the second aperture part and adjust the aperture opening of the annular aperture.

16. The computed tomography system as claimed in claim 2, wherein
the static gantry includes an annular aperture having at least a first aperture part and a second aperture part,
the annular aperture is arranged in front of the beam outlet opening of the X-ray source ring, and
the first aperture part is fastened to the detector ring or is an integral part of the detector ring.

17. The computed tomography system as claimed in claim 4, wherein the detector ring and the X-ray source ring partially overlap in the axial direction.

18. The computed tomography system as claimed in claim 17, wherein
the computed tomography system further includes a patient couch configured to move into and out of the static gantry in the axial direction, and
the detector ring is attached to the static gantry at a side of the patient couch in the axial direction.

19. The computed tomography system as claimed in claim 10, wherein
the housing section includes a scattered beam collimator that is oriented to shield detector elements of the detector ring from scattered beams of the X-ray source ring, and
the housing section is configured such that when the at least one of the functional units is inserted into the housing section, the detector elements of the at least one of the functional units are inserted in a fitting manner with respect to the scattered beam collimator.

20. The computed tomography system as claimed in claim 19, wherein
the detector ring includes multiple housing sections, and
the multiple housing sections, when assembled, define an annular shape of the detector ring.
